(19) United States
(12) Patent Application Publication
(10) Pub. No.: US 2024/0094213 A1
(43) Pub. Date: Mar. 21, 2024

(54) THORACIC DUCT AS A SAMPLE COLLECTION RESERVOIR

(71) Applicant: Washington University, St. Louis, MO (US)

(72) Inventors: Jose P. Zevallos, St. Louis, MO (US); Aadel Chaudhuri, St. Louis, MO (US)

(21) Appl. No.: 17/948,760

(22) Filed: Sep. 20, 2022

Related U.S. Application Data
(60) Provisional application No. 63/246,208, filed on Sep. 20, 2021.

(51) Int. Cl. G01N 33/574; C12Q 1/6886; G01N 33/49; G01N 33/50; G01N 33/68

(52) U.S. Cl. CPC G01N 33/57488; C12Q 1/6886; G01N 33/49; G01N 33/5091; G01N 33/6893; G01N 2800/7028

(57) ABSTRACT

The present invention provides methods of diagnosing diseases and other pathologies by obtaining fluid from a thoracic duct or right lymphatic duct of a subject and detecting one or more biomarkers indicative of a disease or pathology in the collected fluid.

### CROSS-REFERENCE TO RELATED APPLICATIONS

This application claims the benefit of and priority to U.S. Provisional Application No. 63/246,208, filed Sep. 20, 2021, which is incorporated herein by reference in its entirety.

### TECHNICAL FIELD

This invention provides methods and devices for diagnosing disease and/or assessing patient treatment options based on biomarkers identified from lymphatic fluid.

### BACKGROUND

Cancer is a global health issue that causes millions of deaths worldwide every year. To treat cancer, clinicians often resort to chemotherapy, which involves a systemic cocktail of highly toxic drugs. Specific treatment parameters (e.g., drugs used or dosing regimen) are generally based on clinical outcomes across many different patients. However, it is known that individual patient outcomes vary greatly. Consequently, current treatments would benefit some patients while other patients receive little or no benefit or may even suffer from adverse side effects. Personalized medicine has aimed to address this problem.

Personalized medicine focuses on patient-specific disease indicators (biomarkers) to tailor treatment based on an individual's predicted treatment response or risk of disease. The identification of reliable and informative biomarkers is therefore paramount.

Tissue, such as tumor tissue, is generally the most common source of information on disease status and from a histologic standpoint is often considered the gold standard. Unfortunately, tissue samples are often difficult to access and subject to limited availability, especially without performing an invasive procedure. In the context of cancer, often by the time tumors are detected, cancer has spread or progressed. Thus, many conventional attempts to derive information regarding disease progression have focused on identifying molecular biomarkers from bodily fluids, such as blood or urine.

Blood is of high clinical interest because of its accessibility and the ability to identify circulating tumor DNA. However, such blood-based biomarkers are often degraded and are only present in advanced diseases. Thus, the lack of reliable biomarkers continues to inhibit the potential of tailoring therapeutics on an individual basis.

### SUMMARY

This invention provides methods that make use of samples that are conventionally regarded as waste. According to the invention, drain fluid (e.g., from a surgery or investigative assay) that is usually discarded contains detectable biomarkers that are useful for diagnosing pathologies, monitoring disease progression and spread, and assessing therapeutic choice and treatment efficacy. In particular, the invention provides methods of obtaining lymphatic drain fluid from a thoracic duct and/or right lymphatic duct of a subject and detecting relevant biomarkers to provide a diagnostic or prognostic assessment.

Many surgical procedures involve resection, dissection, or excision surgeries that produce lymphatic drain fluid. In addition, as part of a postoperative regime, patients receive an implanted surgical drain, such as a Jackson-Pratt (JP) drain, which removes lymph fluid that collects at the site of a surgery. However, whether lymph fluid has been drained or collected during a surgical intervention, or as part of postoperative recovery, it has traditionally been discarded as biological waste.

The present invention makes use of this drain fluid that is conventionally discarded. According to the invention, drain fluid contains diagnostic biomarkers that are useful for diagnosis, prognosis, therapeutic selection and efficacy. The present invention includes methods in which drain fluid is obtained from a thoracic duct or a right lymphatic duct and using the fluid to assess biomarkers of disease. The biomarker may include, for example, a biomarker associated with cancer, such as tumor cell, cell-free tumor DNA, RNA or protein. Thus, certain methods further include a step of identifying the cancer biomarker in the fluid. Methods may also include a step of quantifying the cancer biomarker. Detecting or quantifying the presence of a cancer biomarker may include sequencing DNA or RNA from a tumor cell or from cell-free tumor DNA, obtained from the fluid. Moreover, detection may comprise a protein detection assay, such as an ELISA (enzyme-linked immunosorbent assay) or others. Biomarker content may be weighted or may be in the form of a ratio to standards or other biomarkers. For example, the ratio of biomarker levels in the duct fluid and a blood sample may be evaluated. In certain aspects, the ratio of a biomarker in duct fluid versus blood is indicative of the stage of disease, wherein a greater amount in duct fluid versus blood is indicative of early-stage disease. In certain embodiments of the invention the lymphatic fluid from the thoracic duct is separated from other fluids prior to detecting diagnostic biomarkers.

In certain embodiments, the fluid from the thoracic duct is obtained as a by-product of a surgery. The surgery may include a resection, dissection, or excision. The surgery may not be a cancer-related surgery, yet it may still provide a lymphatic fluid sample that provides detectable levels of a biomarker indicative of cancer. In certain aspects, the surgical effluent is captured, e.g., through the use of a drain, within 24 hours of a surgical procedure.

The present invention also provides a method to diagnose a disease that includes the steps of obtaining fluid from a thoracic duct of a subject and assaying the fluid from the thoracic duct for a diagnostic biomarker. In certain aspects, the method does not include isolating tumor-associated genetic material from the fluid and/or nucleic acid sequencing.

In certain aspects, a biomarker analyzed by a method of the invention is indicative of a disease other than cancer. In certain methods, an analyzed biomarker includes solid material in the fluid from a thoracic duct.

### BRIEF DESCRIPTION OF THE DRAWINGS

The patent or application file contains at least one drawing executed in color. Copies of this patent or patent application with color drawing(s) will be provided by the Office by request and payment of the necessary fee.

FIG. 1 provides a comparison of methods for staging cancer with and without the insight that fluid collected.

### DETAILED DESCRIPTION

It will be appreciated that for simplicity and clarity of illustration, where appropriate, reference numerals have been repeated among the different figures to indicate corresponding or analogous elements. In addition, numerous specific details are set forth in order to provide a thorough understanding of the examples described herein. However, it will be understood by those of ordinary skill in the art that the examples described herein can be practiced without these specific details. In other instances, methods, procedures and components have not been described in detail so as not to obscure the related relevant feature being described. Also, the description is not to be considered as limiting the scope of the embodiments described herein. The drawings are not necessarily to scale and the proportions of certain parts may be exaggerated to better illustrate details and features of the present disclosure.

Several definitions that apply throughout the above disclosure will now be presented. The terms “comprising,” “including” and “having” are used interchangeably in this disclosure. The terms “comprising,” “including” and “having” mean to include, but not necessarily be limited to the things so described.

This invention is based on the recognition that lymphatic fluid contains detectable biomarkers that can be used for diagnosing pathologies, monitoring disease progression and spread, therapeutic selection, and assessing treatment efficacy. In particular, the invention provides methods of obtaining lymphatic fluid from a thoracic duct and/or right lymphatic duct of a subject and detecting relevant biomarkers to provide a diagnostic or prognostic assessment.

Methods and systems of the invention may be performed in conjunction with a surgical procedure. By way of example, a patient may undergo a surgical procedure, e.g., a resection, dissection, excision, transplant, or reconstructive surgery. Fluid may be collected from the right lymphatic duct or thoracic duct during the surgery and analyzed for biomarkers. In preferred aspects, the fluid analyzed is obtained from a subject via a surgical drain, e.g., a JP drain, which collects fluid from a subject's thoracic duct or the right lymphatic duct. Such drains are often used after a surgical procedure or in response to an illness, which may cause lymphatic fluid levels to increase, cause the fluid to pool, or effuse (e.g., as in a pleural effusion).

Thus, methods and systems of the invention may use a surgical drain device to obtain fluid from the thoracic duct or the right lymphatic duct of a subject. For example, certain methods and systems of the invention collect the fluid using a surgical drain tube, a surgical wound vac, a JP drain, and any other suitable surgical drain known in the art. In certain methods, the fluid is collected using custom surgical drain devices. Such devices, for example, include components configured to preserve the integrity of the biomarker(s) to be studied, e.g., nucleic acids, proteins, and other analytes. Similarly, the devices may include components configured to preserve the integrity of the biomarker(s) source, e.g., extracellular vesicles and cancer cells. The devices may include components used to perform at least a portion of sample preparation steps, and/or any other suitable function related to obtaining, preserving, and processing the fluid sample.

In certain aspects, the biomarker(s) analyzed in fluid collected from a subject include, for example, tumor cells, immune cells, bacterial cells, viral host cells, donor organ cells, microvascular cells, cell-free DNA (cfDNA), cell-free RNA (cfRNA), circulating tumor DNA (ctDNA), messenger RNA, exosomes, proteins, hormones, and analytes. The biomarker(s) analyzed depend on, for example, a specific patient, pathology, surgery type, and surgery site. By analyzing biomarkers in the obtained fluid, methods of the invention provide diagnostic or prognostic information. The biomarker(s) in the thoracic duct or the right lymphatic duct fluid may be isolated using methods suitable to the analyte of interest, for example, filtering, and centrifuging, chromatography. In certain methods, the biomarker includes any one or more of interleukin-1, interleukin-6, interleukin-10, a tumor necrosis factor, matrix metalloproteinase-1, matrix metalloproteinase-2, matrix metalloproteinase-9, matrix metalloproteinase-13, or a nucleic acid comprising a mutation. In some embodiments, the biomarker is a ratio of circulating tumor cells to cell-free DNA detected in the fluid.

In certain aspects, methods include collecting biological samples from a subject in addition to the fluid obtained from the subject's thoracic duct or the right lymphatic duct. For example, the additional sample may include one or more of blood, plasma, urine, a tissue biopsy, and the like. In preferred aspects, the additional sample includes blood or plasma. These samples may be analyzed for biomarkers and/or other features indicative of a subject's diagnosis or prognosis. In certain methods and systems of the invention, combining data derived from the fluid and the additional sample(s) provides a more meaningful assessment. For example, a tissue biopsy may reveal cancer or other localized pathology. Biomarker(s) analyzed in fluid from the thoracic duct or the right lymphatic duct may reveal more high-level or systemic data regarding the pathology revealed in the tissue sample, e.g., an indication if an initial cancer has metastasized. Conversely, the thoracic duct or right lymphatic duct may be proximal to a surgical site, and the derived biomarkers provide a local-level analysis of the pathology/recovery. In such an instance, biomarkers from blood or plasma may provide contrasting systemic information.

In certain aspects, the present invention provides methods to provide a diagnostic or prognostic assessment of a disease by obtaining a fluid sample, e.g., lymph fluid, from a lymphatic duct, and measuring quantities or concentrations of one or more biomarkers in the fluid. The levels of biomarkers measured may be compared with a threshold value or range of values to classify a subject's condition. In certain aspects, the levels of biomarkers in a sample from lymphatic duct fluid are correlated or combined with levels of biomarkers from another sample source, e.g., blood or plasma.

In certain aspects, biomarker concentrations, either exclusively from lymph duct fluid or in combination with other sample types, are combined to produce a score or value to summarize a subject's condition or an aspect of a disease. Measured biomarker concentrations may be manipulated, e.g., summed, subtracted, weighted, multiplied together, ratioed, correlated, etc. to determine a subject's condition. For example, in certain methods of the invention, equal volumes of lymph fluid and blood are collected from a subject after tumor removal. Each sample is analyzed to determine the relative concentrations of cancer cells or nucleic acid molecules specifically correlated to the removed tumor. A larger amount in lymph fluid, when compared to blood, is indicative of the continued presence of disease.

In various aspects, biomarkers may be detected and quantified using methods known in the art. Suitable assays include, for example, nucleic acid sequencing, PCR, quantitative PCR, digital droplet PCR, Western blot target capture, proteomics, nucleic acid expression analysis, antibody screening, and the like.

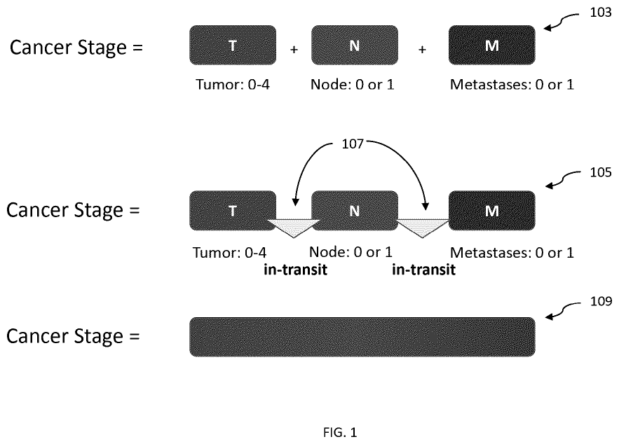

FIG. 1 provides a general example of the value provided by systems and methods of the invention, either when used with or without a combined sample. Cancer staging may include an analysis of various molecular and diagnostic indicators. FIG. 1 provides general schematics for staging or assessing the progression of oropharyngeal cancer. The topmost schematic 103 shows a current paradigm for staging the cancer. As shown, the cancer stage or progression is determined by combining three scores, i.e., the tumor score, the node score, and the metastases score.

The tumor score may include components such as the size, location, and histopathologic components detected in a tumor sample. In order to calculate the tumor score, it is often necessary to conduct an invasive procedure, e.g., via a tissue biopsy or a resection surgery. In the example provided in FIG. 1, the node score is binary, which may indicate the presence or absence of positive lymph nodes (nodes with cancer cells). Like the tumor score, this generally requires an invasive process to remove a lymph node for study. Often, for oropharyngeal cancer, this occurs during a neck resection surgery or a similar procedure. The metastases score is likewise binary. In the context of oropharyngeal cancer, this often involves detecting extranodal extensions (ENE).

FIG. 1 also provides a generalized schematic 105 incorporating methods and systems of the invention, by way of example, to stage oropharyngeal cancer. The triangles 107 between the tumor, node, and metastases scores indicate time periods at which fluid may be obtained from a subject's thoracic duct or the right lymphatic duct, and analyzed for biomarkers indicative of the oropharyngeal cancer stage. Although fluid may be collected at various time points to successfully analyze pathologies, the times chosen in this example represent likely periods after a surgical procedure, e.g., tumor and/or lymph node removal surgeries. Often, these surgeries are accompanied by a postoperative surgical drain from which the analyzed fluid may be obtained without any additional risk or discomfort to the subject. The fluid was already going to be collected and disposed of as medical waste.

As shown in FIG. 1, biomarkers collected at these time points may, for example, detect biomarkers indicative of a tumor's spread, or potential to spread, into the lymph nodes and/or metastasize beyond the lymph system. For example, in the context of oropharyngeal cancer, the present Inventors discovered that lymph fluid contained detectable levels of circulating tumor human papillomavirus DNA (ctHPVDNA) could be used to diagnose, stage, and predict clinical treatment efficacy and outcomes. Thus, the methods of the invention may provide more detailed and accurate analyses of many pathologies. As shown, the biomarkers, including when combined with other data or samples (e.g., the tumor, node, and/or metastases scores), may provide a continuous assessment and prediction 109 of a disease's progression in a subject.

The presently-disclosed methods and systems may be used to perform longitudinal analysis of a subject. Fluid obtained from a subject's thoracic duct or the right lymphatic duct may be collected at a number of time points. This generally occurs using a surgical drain, which remains in the patient and continually collects excess fluid, e.g., lymph fluid, from the subject's thoracic duct or the right lymphatic duct. The drain may be inserted into the patient solely to collect fluid samples. However, surgical drains often accompany surgical procedures or in order to drain fluid caused by an injury or infection—time periods when assessing a subject's condition over time are of pressing concern. Advantageously, analyzing the collected fluid over time puts no more burden on the subject, as the fluid is otherwise disposed as waste. Thus, without inducing further injury on the patient, the presently disclosed systems and methods may provide longitudinal insights into a subject's recovery, the efficacy of an administered treatment, and the progression of the disease.

For example, in certain aspects, the disclosed systems and methods are used following a surgical procedure for the treatment of cancer, e.g., a tumor removal. Fluid, e.g., lymph fluid, is collected using a drain inserted after the surgery. Biomarkers in the collected fluid are used to provide an assessment of whether the tumor removal removed all cancer or if the cancer is continuing to grow and/or show signs of spreading. In certain aspects, the biomarkers in the fluid are analyzed in conjunction with other bioassay data, e.g., histopathology results, genetic data, and biomarkers in blood or plasma. In certain aspects, the methods of the invention replace or supplement medical imaging or other diagnostic methods used by a practitioner to perform a post-surgical restaging of a subject's cancer, determine the subject's prognosis, select a particular adjuvant treatment, and/or assess treatment efficacy.

Methods of the invention provide an avenue for non-invasive, postoperative disease management by evaluating by-products collected from lymphatic ducts that drain fluid from peripheral tissues proximal to the site of diseased or wounded tissue, e.g., the site of a tumor removal. Fluid recovered from a surgical drain may contain material informative of an excised tumor as well as the milieu that surrounded the tumor. Accordingly, the invention recognizes that lymphatic fluid is of high clinical interest not only because of its relevance to a subject's tumor but also for the insight it provides into the physiological conditions that gave rise to the tumor. Biomarkers collected from lymphatic fluid near a site of an excised tumor can inform on a patient's immune and/or inflammatory response following the tumor resection. Quantities of certain biomarkers, and combinations thereof, can be correlated with known patient outcomes to determine a disease prognosis. Accordingly, methods of the invention can use biomarkers collected from lymphatic fluid to detect residual disease or determine whether the disease is likely to recur.

In some instances, methods of the invention involve collecting lymphatic for the purpose of obtaining a sample. The fluid can be collected by intubating a lymphatic vessel of a patient and draining the lymphatic fluid into a collection vessel. The lymphatic fluid can be collected for the purpose of identifying biomarkers indicative of cancer. Alternatively, the lymphatic fluid is collected during a procedure that is unrelated to cancer, and as such, the lymphatic fluid may be a by-product of an unrelated intervention. Accordingly, collection of lymphatic fluid in some instances does not impose any additional inconvenience to a patient or clinician. And since the lymphatic system involves an extensive network of vessels throughout the human body, the lymphatic system can provide a source of diagnostic material of a pathology, such as a tumor, despite its actual location within the body.

Certain methods and systems of the invention provide the ability to diagnose a disease by detecting the presence or absence of one or more biomarkers indicative of the disease in fluid obtained from a lymphatic duct. Detected biomarkers collected from the fluid may provide data to diagnose a disease or to assess disease severity. For example, quantities of certain biomarkers, or combinations of biomarkers, identified from the fluid may be correlated with a known clinical outcome or a treatment response. As such, methods of the invention are useful to identify an optimal treatment for a patient or identify aggressive treatments that can be avoided, such as chemotherapy.

In certain aspects, the lymphatic duct fluid is obtained during a surgery. However, the scope of the invention is not limited to fluid from any one type of surgery. The surgery can be any form of bodily intervention, including an intervention that is wholly unrelated to a disease diagnosed using the methods of the invention. As an example, the fluid can be collected while treating edema caused by an allergic reaction. In some preferred embodiments, the fluid is produced and collected in response to a resection surgery. The fluid may be collected as early as twelve to twenty-four hours following surgery. Fluid collection may extend for a number of days and weeks to assure proper fluid draining and/or obtaining a desired number of samples over time.

In preferred aspects, the collected fluid includes lymphatic fluid, lymphovascular fluid, insterstitial fluid, or any combination thereof. Lymphatic fluid contains waste products, including cells, cellular debris, bacteria, protein, and nucleic acid. It is an insight of the invention that an analysis of these waste products can inform on disease. Some embodiments of the invention include separating lymphatic fluid, or components thereof, from drain fluid. According to aspects of the invention, the separated portion of drain fluid will contain a greater quantity of biomarkers than can be obtained from an equal volume of blood.

Methods of the invention are useful to detect a health condition even before clinical symptoms appear. In certain aspects, the biomarkers reveal residual disease as well as factors that led to disease formation, e.g., tumor growth, which is useful to assess the risk of disease recurrence. An analysis of biomarkers for immune or inflammatory response provides useful data to identify a disease prognosis.

In certain aspects, fluorescent labels may be used to identify biomarkers analyzed in the systems and methods of the invention. A fluorescent label or fluorescent probe is a molecule that is attached chemically to aid in the detection of a biomarker. Fluorescent labeling generally uses a reactive derivative of a fluorescent molecule known as a fluorophore. The fluorophore selectively binds to a specific region or functional group on the biomarker and may be attached chemically or biologically. Any known technique for fluorescent labeling may be used, for example, enzymatic labeling, protein labeling, or genetic labeling. Any known fluorophore may also be used. Both the fluorophore and labeling techniques may be selected and adjusted based on the biomarker to be identified. The most commonly labeled molecules are antibodies, proteins, amino acids, and peptides which are then used as specific probes for detection of a particular target.

Fluorescent labeling may be used to identify and quantify a biomarker in a lymphatic duct sample without separating the components of the fluid. In certain methods, by providing fluorescent labels directly into the fluid, fluorescent microscopy or a colorimetric assay may be used to identify and quantify the presence of the biomarker from a color change alone. For example, fluorescent labels may be applied to the fluid during a post-operative period to provide valuable information to a practitioner regarding a subject's condition.

When quantifying a biomarker, barcodes may be added to a biomarker to aid in amplification, detection, or differentiation of the biomarker. Barcodes may be added to biomarkers by “tagging” the biomarker with the barcode. Tagging may be performed using any known method for barcode addition, for example, direct ligation of barcodes to one or more of the ends of a nucleic acid molecule or protein. Nucleic acid molecules may, for example, be end repaired in order to allow for direct or blunt-ended ligation of the barcodes. Barcodes may also be added to nucleic acid molecules through first or second strand synthesis, for example using capture probes or primers. First and second strand synthesis may be used in RNA analysis to generate tagged DNA molecules.

Unique molecular identifiers (UMI) are a type of barcode that may be provided to biomarkers in a sample to make each biomarker, together with its barcode, unique, or nearly unique. For example, with regard to nucleic acid molecules, this is accomplished by adding, e.g. by ligation or reverse transcription, one or more UMIs to each nucleic acid molecule such that it is unlikely that any two previously identical nucleic acid molecules, together with their UMIs, have the same sequence. By selecting an appropriate number of UMIs, every nucleic acid molecule in the sample, together with its UMI, will be unique or nearly unique. One strategy for doing so is to provide to a sample of nucleic acid molecules a number of UMIs in excess of the number of starting nucleic acid molecules in the sample. By doing so, each starting nucleic molecule will be provided with different UMIs, therefore making each molecule together with its UMIs unique.

UMIs are also advantageous in that they can be useful to correct for errors created during amplification, such as amplification bias or incorrect base pairing during amplification. For example, when using UMIs, because every nucleic acid molecule in a sample together with its UMI or UMIs is unique or nearly unique, after amplification and sequencing, molecules with identical sequences may be considered to refer to the same starting nucleic acid molecule, thereby reducing amplification bias. Methods for error correction using UMIs are described in Karlsson et al., 2016, “Counting Molecules in cell-free DNA and single cells RNA”, Karolinska Institutet, Stockholm Sweden, the contents of which are incorporated herein by reference.

For RNA or mRNA sequencing, sequencing may first include the steps of preparing a cDNA library from barcoded RNA, for example through reverse transcription, and sequencing the cDNA. cDNA sequencing may advantageously allow for the quantification of gene expression within the single cell and can be useful to identify characteristics of the single cell to, for example, make a diagnosis, prognosis, or determine drug effectiveness.

Reverse transcription may be performed using, without limitation, dNTPs (mix of the nucleotides dATP, dCTP, dGTP, and dTTP), buffer/s, detergent/s, or solvent/s, as required, and suitable enzyme such as polymerase or reverse transcriptase. The polymerase used may be a DNA polymerase, and may be selected from Taq DNA polymerase, Phusion polymerase (as provided by Thermo Fisher Scientific, Waltham, Massachusetts), or Q5 polymerase. Nucleic acid amplification reagents are commercially available, and may be purchased from, for example, New England Biolabs, Ipswich, MA, USA. The reverse transcriptase used in the presently disclosed targeted library preparation method may be, for example, maxima reverse transcriptase.

Reverse transcription may be performed by oligos that have a free, 3′ poly-T region. The 3′ portions of the cDNA capture oligos may include gene-specific sequences or oligomers, for example, capture primers to reverse transcribe RNA guides comprising a capture sequence. The oligomers may be random or “not-so-random” (NSR) oligomers (NSROs), such as random hexamers or NSR hexamers. The oligos may include one or more handles such as primer binding sequences cognate to PCR primers that are used in the amplifying step or the sequences of NGS sequencing adaptors. The reverse transcription primers may include template switching oligos (TSOs), which may include poly-G sequences that hybridize to and capture poly-C segments added during reverse transcription.

Reverse transcription of non-polyadenylated RNA may comprise the use of a capture sequence and a capture primer or probe. Primer sequences may comprise a binding site, for example, a primer sequence that would be expected to hybridize to a complementary sequence, if present, on any nucleic acid molecule released from a cell and provide an initiation site for a reaction. The primer sequence may also be a “universal” primer sequence, i.e. a sequence that is complementary to nucleotide sequences that are very common for a particular set of nucleic acid fragments. Primer sequences may be P5 and P7 primers as provided by Illumina, Inc., San Diego, California. The primer sequence may also allow a capture probe to bind to a solid support.

Reverse transcription can also be useful for adding a barcode or a UMI, or both to cDNA. This process may comprise hybridizing the reverse transcription primer to the probe followed by a reverse transcription reaction. The complement of a nucleic acid when aligned need not be perfect; stable duplexes may contain mismatched base pairs or unmatched bases. Those skilled in the art of nucleic acid technology can determine duplex stability empirically considering a number of variables including, for example, the length of the oligonucleotide, percent concentration of cytosine and guanine bases in the oligonucleotide, ionic strength, and incidence of mismatched base pairs.

Nucleic acid molecules may advantageously be amplified prior to sequencing. Amplification may comprise methods for creating copies of nucleic acids by using thermal cycling to expose reactants to repeated cycles of heating and cooling, and to permit different temperature-dependent reactions (e.g. by Polymerase chain reaction (PCR). Any suitable PCR method known in the art may be used in connection with the presently described methods. Non-limiting examples of PCR reactions include real-time PCR, nested PCR, multiplex PCR, quantitative PCR, or touchdown PCR.

Sequencing nucleic acid molecules may be performed by methods known in the art. For example, see, generally, Quail, et al., 2012, A tale of three next generation sequencing platforms: comparison of Ion Torrent, Pacific Biosciences and Illumina MiSeq sequencers, BMC Genomics 13:341. Nucleic acid molecule sequencing techniques include classic dideoxy sequencing reactions (Sanger method) using labeled terminators or primers and gel separation in slab or capillary, or preferably, next generation sequencing methods. For example, sequencing may be performed according to technologies described in U.S. Pub. 2011/0009278, U.S. Pub. 2007/0114362, U.S. Pub. 2006/0024681, U.S. Pub. 2006/0292611, U.S. Pat. Nos. 7,960,120, 7,835,871, 7,232,656, 7,598,035, 6,306,597, 6,210,891, 6,828,100, 6,833,246, and 6,911,345, each incorporated by reference.

The conventional pipeline for processing sequencing data includes generating FASTQ-format files that contain reads sequenced from a next generation sequencing platform, aligning these reads to an annotated reference genome, and quantifying the expression of genes. These steps are routinely performed using known computer algorithms, which a person skilled in the art will recognize can be used for executing the steps of the present invention. For example, see Kukurba, Cold Spring Harb Protoc, 2015 (11):951-969, incorporated by reference.

### EXAMPLES

### Example 1

After neck resection surgery to remove an oropharyngeal tumor, subjects receive an implanted surgical drain, e.g., a JP drain. 24 hours post-surgery, fluid is collected from a lymphatic duct from each subject via the drain. Each fluid sample is centrifuged and filtered. A nuclease, such as EDTA is added to each sample.

Biomarkers associated with oropharyngeal cancer are isolated and measured from the samples, which include tumor-associated genetic material. The tumor-associated genetic material includes, for example, one or more of cell-free nucleic acids, nucleic acids from a tumor, nucleic acids from an isolated exosome, and/or viral nucleic acids.

Once isolated, the tumor-associated genetic material is analyzed using one or more of nucleic acid sequencing, PCR, and/or Western blot.

This analysis of tumor-associated genetic material provides results that may include, for example, quantities of detected nucleic acids, mutations, variants, copy number, and expression patterns. These results may be compared with other bioassay results, either for other biomarkers in the fluid from the lymphatic duct and/or from a different sample type, such as blood or plasma.

Using these results, one or more scores are produced indicative of the subjects' conditions, disease states, and prognosis. The scores provide a practitioner with valuable insight as to whether to pursue additional therapeutic intervention, e.g., additional surgery, medications, and active monitoring.

### INCORPORATION BY REFERENCE

References and citations to other documents, such as patents, patent applications, patent publications, journals, books, papers, web contents, have been made throughout this disclosure. All such documents are hereby incorporated herein by reference in their entirety for all purposes.

### EQUIVALENTS

Various modifications of the invention and many further embodiments thereof, in addition to those shown and described herein, will become apparent to those skilled in the art from the full contents of this document, including references to the scientific and patent literature cited herein. The subject matter herein contains important information, exemplification and guidance that can be adapted to the practice of this invention in its various embodiments and equivalents thereof.

What is claimed is:

1. A method of disease diagnosis, the method comprising the steps of:
obtaining fluid from a thoracic duct or a right lymphatic duct of a subject; and
detecting a cancer biomarker in the fluid.

2. The method of claim 1, wherein the cancer biomarker is a tumor cell or cell-free tumor DNA.

3. The method of claim 1, further comprising the step of identifying the cancer biomarker.

4. The method of claim 3, further comprising the step of quantifying the cancer biomarker.

5. The method of claim 1, further comprising the step of detecting the presence of the cancer biomarker in a blood sample from the subject.

6. The method of claim 5, further comprising the step of evaluating a ratio of the cancer biomarker in the fluid from the thoracic duct and the blood sample.

7. The method of claim 6, wherein if the subject is afflicted with cancer, the fluid from the thoracic duct comprises a greater volume of the cancer biomarker than the same volume of blood obtained from the subject.

8. The method of claim 1, wherein the fluid from the thoracic duct comprises lymphatic fluid.

9. The method of claim 1, wherein the step of detecting the presence of a cancer biomarker comprises sequencing DNA or RNA from a tumor cell or from cell-free tumor DNA.

10. The method of claim 1, further comprising the step of separating lymphatic fluid from the fluid from the thoracic duct.

11. The method of claim 10, where the step of detecting a cancer biomarker comprises detecting the cancer biomarker in the lymphatic fluid.

12. The method of claim 1, wherein the fluid from the thoracic duct is obtained as a by-product of a surgery.

13. The method of claim 12, wherein the surgery comprises a resection, dissection, or excision.

14. The method of claim 12, wherein the surgery is not a cancer-related surgery.

15. The method of claim 12, wherein the surgical effluent is captured within 24 hours of the surgery.

16. A method of disease diagnosis, the method comprising the steps of:
obtaining fluid from a thoracic duct of a subject; and
assaying the fluid from the thoracic duct for a marker of a medical condition, wherein the method does not include isolating tumor-associated genetic material from the fluid.

17. The method of claim 16, wherein the assaying step does not include sequencing.

18. The method of claim 16, wherein the marker of a medical condition is not a marker of cancer.

19. The method of claim 16, wherein the marker comprises solid material in the fluid from the thoracic duct.

20. The method of claim 16, wherein the fluid from the thoracic duct comprises lymphatic fluid.
